(19) United States
(12) Patent Application Publication
(10) Pub. No.: US 2026/0293865 A1
(43) Pub. Date: Oct. 1, 2026

(54) SINGLE-SEX CHICKEN WITH TOXIN-ANTITOXIN SYSTEM

(71) Applicant: New York University, New York, NY (US)

(72) Inventors: Teresa DAVOLI, New York, NY (US); Jef BOEKE, New York, NY (US); Pan CHENG, New York, NY (US)

(21) Appl. No.: 19/489,135

(22) PCT Filed: May 30, 2024
(86) PCT No.: PCT/US2024/031571

§ 371 Date: Dec. 1, 2025

Related U.S. Application Data
(60) Provisional application No. 63/505,806, filed on Jun. 2, 2023.

(51) Int. Cl. A01K 67/0278; C12N 15/85

(52) U.S. Cl. CPC A01K 67/0278; C12N 15/8509; A01K 2217/052; A01K 2227/30; A01K 2267/01; A01K 2267/02; C12N 2015/8518; C12N 2830/002; C12N 2830/34

(57) ABSTRACT

Provided are compositions and methods for producing modified avian animals such as modified female chickens. The modified female chickens have a modified Z chromosome that includes a sequence encoding a toxin, expression of which is under control of a promoter configured so that expression of the toxin is lethal to an oocyte, zygote or embryo that has the Z′ chromosome. The modified Z′ chromosome may also include a sequence encoding an anti-toxin that mitigates lethality of the toxin to permit development of a male chicken from an egg fertilized by the oocyte or a zygote that has the Z′ chromosome.

### CROSS-REFERENCE TO RELATED APPLICATIONS

This application claims the benefit of U.S. provisional Application no. 63/505,806, filed Jun. 2, 2023, the entire disclosure of which is incorporated herein by reference.

### FIELD

The present disclosure is related to compositions and methods that selectively facilitate production of female avian animals.

### RELATED INFORMATION

Chicken eggs are major sources of protein for human consumption worldwide. The U.S. is the second-largest egg-producing country, providing 96.9 billion eggs in 2020. However, male chicks are killed on the day they are born, because they cannot lay eggs and they are not the breed used for meat. More than 260 million male chicks are killed each year in the U.S. mainly by maceration with a high-speed grinder. This widely unknown practice (chick culling) is challenging the animal welfare ethic and being sharply criticized. Given the ethical concerns, France and Germany banned all chick culling from Jan. 1, 2022, and called on other countries to do the same. Therefore, it is a trend to replace the chick culling with more humane technologies. In addition to ethical concerns, it is also time-consuming and expensive to distinguish the sex of chicks, which require well-trained workers to check the genital organ or feathers of one-day-old chicks one by one. Newly developed in-ovo sexing methods, determining the sex while chicks are still inside the eggs to avoid the ethical concerns of chick culling, require specialized and costly instruments such as spectroscopes or MRI scanners. Therefore, animal-friend and economic technologies are urgently needed and of great economic value. The present disclosure is related to this need.

### BRIEF SUMMARY

This disclosure provides female chickens comprising a modified Z chromosome (Z′) that comprises a sequence encoding a toxin that is operably linked to a promoter such that activation of transcription by the promoter and concomitant expression of the toxin is lethal to an oocyte, zygote or embryo comprising the Z′ chromosome carried by the chicken. The toxin may also be lethal to an oocyte comprising the Z′ chromosome. The promoter that regulates expression of the toxin comprises the promoter of a gene such that transcription from the promoter occurs during an oocyte, zygote, or embryonic stage of development. In an example, the Z′ chromosome is further modified to comprise a sequence encoding an anti-toxin that mitigates lethality of the toxin. The sequence encoding the anti-toxin is operably linked to an inducible promoter which is configured so that induction of transcription from the inducible promoter and expression of the anti-toxin permits development of a male chicken from an egg fertilized by the oocyte or a zygote that comprises the Z′ chromosome. In non-limiting examples, the toxin comprises barnase and the anti-toxin comprises barstar. The disclosure includes eggs obtained from a modified chicken that comprises the described modifications.

The disclosure also provides a method of making a female chicken that is capable of selectively producing only female offspring. This method comprising modifying the Z′ chromosome of a parental female chicken to include a sequence encoding a toxin that is operably linked to a promoter such that activation of transcription by the promoter and expression of the toxin is lethal to an oocyte, zygote, or early embryo comprising the Z′ chromosome carried by the chicken. The promoter is configured to regulate expression of the toxin such that transcription from the promoter only occurs during an oocyte, zygote, or an early embryonic stage of development. This method can also include further modifying the Z′ chromosome of the parental chicken to comprise a sequence encoding an anti-toxin that mitigates lethality of the toxin. The sequence encoding the anti-toxin is operably linked to an inducible promoter, such that induction of the inducible promoter and expression of the anti-toxin permits development of a chicken that contains the Z′ chromosome. This example allows perpetuation of male and female progeny and perpetuation of the line of chickens.

The disclosure also provides a method of selectively breeding only female chickens by mating a parental chicken with the described genetic modifications with a normal male chicken. The male zygotes or embryos of genotype Z′Z are non-viable, thereby producing only female chickens. The disclosure also provides for mating

a chicken with the described genetic modifications with a male chicken and inducing expression of the anti-toxin to thereby produce viable male and female offspring to maintain a breeding flock.

The disclosure also provides a kit comprising one or more containers that contain one or more described polynucleotides. The polynucleotides may be provided as isolated polynucleotides, or the polynucleotides may be components of any suitable expression vector or system, such as viral expression vectors.

### BRIEF DESCRIPTION OF THE FIGURES

FIG. 1 provides an overview of embodiments of this disclosure, illustrated using a representative and non-limiting toxin-antitoxin combination.

FIG. 2 provides the schematic of a proof-of-concept experiment performed in Chicken Embryonic Fibroblasts (CEFs) cells designed to show the effectiveness of the toxin (Barnase) anti-toxin (Barstar) approach in killing chicken cells in a selective manner, i.e., CEFs are able to express Barnase only in the presence of the antitoxin Barstar, indicating that Barnase is toxic in the absence of Barstar. Panel A represents the schematics of the experiment, and the results are presented in panel B.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all genetic constructs, all modified cells, all modified non-human animals, and eggs produced by modified female avian animals, progeny of modified parental female chickens and male chickens, and all method steps described herein.

In one approach the present disclosure provides compositions and methods used for selective elimination of male non-human animals, such as male avian animals, by inhibiting development of non-human male avian animals. Inhibition of development of non-human male animals can be achieved by genetic modifications that render oocytes incapable of viable fertilization, or are lethal to a male zygote or male embryo.

In non-limiting examples, non-human animals that are modified to selectively produce only female offspring are avian animals. In examples the avian animals are Galliformes and thus include any members of the order of heavy-bodied ground-feeding birds that includes turkey, grouse, chicken, New World quail and Old World quail. In examples, the avian animals are domesticated fowl, including but not limited to domesticated chickens and turkeys. In examples, the chickens are Gallus gallus, such as Gallus gallus domesticus. In examples, the chickens are broiler chickens.

With respect to chickens in general, their gender is determined by sex chromosomes (Z and W chromosomes). Male chickens carry two Z chromosomes (ZZ), while female chickens have a Z and a W chromosome (ZW). Therefore, female chickens can produce oocytes carrying either a Z chromosome or a W chromosome, while male chickens can only generate sperm having a Z chromosome. Accordingly, the gender of offspring is determined by the sex chromosome inherited from female chickens.

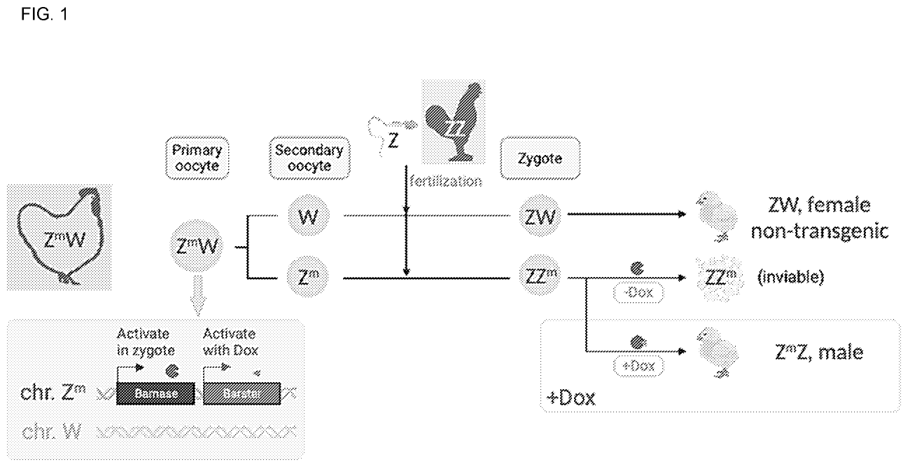

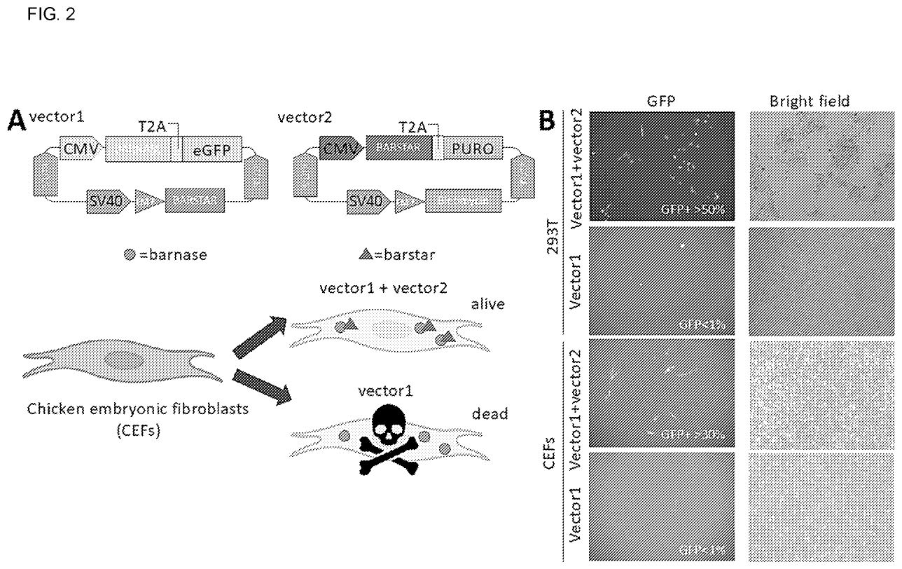

In a non-limiting approach, a presently described method selectively eliminates zygotes inheriting a Z chromosome from hens (ZZ, male zygotes) without influencing zygotes with a W chromosome from hens (ZW, female zygotes). As an example, as illustrated by FIG. 1, the disclosure includes a parental female chicken that has a modified Z chromosome (referred to as “Z′”) that comprises a sequence encoding a toxin that is operably linked to a promoter. By “operably linked” it is meant that the promoter is configured to drive transcription of a toxin-encoding mRNA. In examples, the promoter is a promoter that it is only active in an oocyte, zygote or embryo, such as an early stage of embryo development. Early stages of chicken embryo development include, for example, the first appearance of embryonic tissue, before the appearance of the primitive streak, or before the first appearance of blood vessels. As such, cells that are not eliminated by the toxin are exclusively female, and permit development of a hatched chick that does not have chromosomes that are modified to express the toxin or encode the antitoxin, whereas all exogenous DNA is only inherited by male zygotes. In examples, the selective prevention of development of male chickens is reversible by using a described toxin-antitoxin system, as represented by the non-limiting illustration in FIG. 1. FIG. 1 shows activation of a representative toxin gene by a promoter that is activated during the zygote stage of development. Likewise, FIG. 2 provides an overview of genetic modifications that are made to female parental chickens, and use of the constructs to selectively produce only female chickens, and to allow mating of modified parental female chickens and male chickens.

FIG. 2 provides a proof-of-concept validation of Barnase-Barstar regulated cell death in Chicken Embryonic Fibroblasts (CEFs). Panel A represents the schematics of the experiment, and the results are presented in panel B. A first lentiviral vector (vector 1) is built to express Barnase linked co-expressing GFP. A second lentiviral vector (vector 2) is built to express Barstar. Lentiviral particles are obtained from the two vectors and used to infect human 293T cells or CEFs. Two types of infections are performed: either with just vector 1 or with both vector 1 and vector 2 using the same amount of vector 1 virus (FIG. 2A). Microscopy-based imaging allows to assess the percentage of GFP-positive cells. The percentage of GFP-positive (GFP+) cells were significantly higher in the cells infected with vector 1 and vector 2 compared to cells infected with only vector1 (FIG. 2B), both in 293Ts and in CEFs.

While lentiviral approaches are used to illustrate examples of this disclosure, other expression vectors may be used, such as adenovirus and adeno associated virus vectors.

In non-limiting examples, the promoter that drives expression of the toxin is activated shortly after fertilization, and may be therefore only temporarily activated in zygotes. As such, the promoter may be involved in zygotic genome activation (ZGA) in so-called “waves.” Non-limiting examples of genes that are under precise development timing in chickens, and therefore have suitable operably linked promoters for expression of the toxin, include the promoters that control expression of genes DLX6, GATA2, ZIC4, WNT11, WNT3A, and C8ORF22. The sequences of these genes are known in the art. In alternative examples, the promoter that drives expression of the toxin is only activated in an early embryonic stage of development, or is only active after meiosis I and before the meiosis Il phase of oocyte development.

The disclosure further comprises use of a gene encoding an anti-toxin under the control of an operably linked inducible promoter. When the operably linked inducible promoter is induced, the anti-toxin is produced, the toxin is neutralized, and allows production of male chicks that carry the Z′ chromosome. This example enables maintenance of parental transgenic chickens for maintaining a breeding flock. Any inducible promoter may be used for this purpose, a non-limiting embodiment of which includes a TET on-inducible promoter, which is inducible by doxycycline. Alternatively, camphor-mediated gene expression regulation, or other inducible promoters can be used. In examples, representative genetic elements that may be used to control gene expression of the described toxin and anti-toxin are described in U.S. Patent Publication No. 2019/0338294, and in U.S. Pat. No. 10,093,938, from which the entire descriptions are incorporated herein by reference.

A non-limiting example of a toxin anti-toxin pair comprises the toxin barnase and the anti-toxin barstar, the amino acid and coding sequences of which are known in the art. Other toxin-antitoxin systems may be substituted, so long as they are under the described developmental gene expression regulation. In an example, a Barstar used in a method of the disclosure has the amino acid sequence that is available under GenBank accession no. AAC53661.1, from which the amino acid sequence is incorporated herein by reference as it exists in the database on the filing date of this application. In an example, the Barnase amino acid sequence is modified such that it has one or both mutations to partially reduce its activity, said mutations being K27 to A and L90 to P. In an example, the BARSTAR used in a method of the disclosure has the amino acid sequence that is available under GenBank accession no. AAP41138.1 from which the amino acid sequence is incorporated herein by reference as it exists in the database on the filing date of this application.

In examples, alternative toxin anti-toxin systems may include a SocB toxin and a SocA antitoxin, or a configuration where the antitoxin is an antisense RNA that binds to a toxin mRNA, or a ribozyme that degrades the mRNA encoding the toxin, or an antitoxin that binds to the target of the toxin and is used to prevent the toxin from causing its lethal effect, or the antitoxin is a protein with ribonuclease activity that specifically cleaves the toxin mRNA.

The disclosure includes polynucleotide constructs described herein, and methods of making parental female chickens that are capable of selectively producing only female offspring, as discussed above. As such, a single DNA cassette, or two DNA cassettes, may be introduced into chicken cells such that the introduced toxin gene is under control of a described developmentally related promoter, which may be endogenous to the chicken, and the anti-toxin coding gene is under the control of an inducible promoter that is also introduced into cells of the chicken.

The disclosure includes kits that include one or more described expression vectors, viral particles, and/or DNA constructs as described herein. The kits may be provided with contents held within one or more preferably sterile containers. The kits may be provided with packaging which may include printed material. The printed material may provide instructions for using the kit components to produce modified chicken cells and modified chickens as described herein.

The DNA cassette(s) can be introduced into avian cells using any suitable compositions and methods used for gene editing, which include but are not necessarily limited to guide-RNA directed nucleases, piggyBac transposon systems, zinc finger nucleases, transcription activator-like effector nucleases (TALENs), and any other designer nuclease that can modify a selected chromosome location. The described cassette(s) can be stably integrated into the germline DNA or maintained as an episomal element that is present during a described developmental stage. The described cassettes can be introduced using any suitable procedure and compositions, including but not limited to electroporation of DNA, viral vector delivery, and the like. The disclosure therefore provides for introducing DNA into parental female chickens that provides for precise spatiotemporal control of toxin and anti-toxin production during predetermined stages of avian development.

The disclosure includes use of recombination recognition sites and cognate recombinases to remove any described genetic element from chicken cells.

The disclosure includes eggs obtained from female chickens that are from a brood of exclusively female chickens produced as described herein.

The disclosure includes selectively breeding only female chickens by mating a described modified parental female chicken with a normal male chicken so that embryos of genotype Z′Z are non-viable, thereby producing only female chickens which do not include any introduced genetic information. A ‘normal” male chicken may be any male chicken that carries a genome that has not been modified.

The disclosure also includes mating a described modified parental female chicken with a normal male chicken, and inducing expression of the anti-toxin to thereby produce viable male and female offspring, such as to maintain a breeding flock. A breeding flock produced according to the described methods is also included in the disclosure.

While the disclosure has been described through specific embodiments, routine modifications will be apparent to those skilled in the art and such modifications are intended to be within the scope of the present disclosure.

1. A female chicken comprising a modified Z chromosome (Z′) that comprises a sequence encoding a toxin that is operably linked to a promoter such that activation of transcription by the promoter and concomitant expression of the toxin is lethal to an oocyte, zygote or embryo comprising the Z′ chromosome carried by the chicken, or is lethal to an oocyte comprising the Z′ chromosome, and wherein the promoter regulating the expression of the toxin is the promoter of a gene wherein transcription from the promoter occurs during an oocyte, zygote, or embryonic stage of development.

2. The female chicken of claim 1, wherein the Z′ chromosome further comprises a sequence encoding an anti-toxin that mitigates lethality of the toxin, and wherein the sequence encoding the anti-toxin is operably linked to an inducible promoter, such that induction of transcription from the inducible promoter and concomitant expression of the anti-toxin permits development of a male chicken from an egg fertilized by the oocyte or a zygote that comprises the Z′ chromosome.

3. The female chicken of claim 1, wherein the toxin comprises barnase.

4. The female chicken of claim 2, wherein the anti-toxin comprises barstar.

5. The female chicken of claim 1, wherein the toxin comprises barnase and the anti-toxin comprises barstar.

6. Eggs obtained from a chicken of claim 1.

7. A method of making a female chicken that is capable of selectively producing only female offspring, the method comprising modifying the Z′ chromosome of a parental female chicken to comprise a sequence encoding a toxin that is operably linked to a promoter such that activation of transcription by the promoter and concomitant expression of the toxin is lethal to an oocyte, zygote, or early embryo comprising the Z′ chromosome carried by the chicken, and wherein the promoter regulating the expression of the toxin is the promoter of a gene such that transcription from the promoter only occurs during an oocyte, zygote, or an early embryonic stage of development.

8. The method of claim 7, further comprising modifying the Z′ chromosome of the parental chicken to comprise a sequence encoding an anti-toxin that mitigates lethality of the toxin, and wherein the sequence encoding the anti-toxin is operably linked to an inducible promoter, such that induction of the inducible promoter and concomitant expression of the anti-toxin permits development of a chicken that contains the Z′ chromosome, allowing perpetuation of male and female progeny and perpetuation of the line of chickens.

9. A method of selectively breeding only female chickens comprising mating a parental chicken of claim 1 with a normal male chicken, and wherein male zygotes or embryos of genotype Z′Z are non-viable, thereby producing only female chickens.

10. Eggs obtained from a chicken produced according to claim 9.

11. A method comprising mating a chicken of claim 5 with a male chicken and inducing expression of the anti-toxin to thereby produce viable male and female offspring to maintain a breeding flock.

12. A kit comprising one or more containers that contain one or more polynucleotides, wherein the one or more polynucleotides comprise a sequence encoding a toxin that is operably linked to a promoter configured such that activation of transcription by the promoter and concomitant expression of the toxin is lethal to an avian oocyte, zygote or embryo comprising a Z′ chromosome, or is lethal to an oocyte comprising the Z′ chromosome, and wherein the promoter regulating the expression of the toxin comprises the promoter of a gene such that transcription from the promoter occurs during an oocyte, zygote, or embryonic stage of development, the kit optionally further comprising a sequence encoding an anti-toxin that mitigates lethality of the toxin, and wherein the sequence encoding the anti-toxin is operably linked to an inducible promoter, such that induction of transcription from the inducible promoter and concomitant expression of the anti-toxin permits development of a male avian animal from an egg fertilized by the oocyte or a zygote that comprises the Z′ chromosome.

13. The kit of claim 12, wherein the toxin comprises barnase.

14. The kit of claim 12, wherein the anti-toxin comprises barstar.

15. The kit of claim 12, wherein the toxin comprises barnase and the anti-toxin comprises barstar.
